(19) United States
(12) Patent Application Publication
(10) Pub. No.: US 2016/0235648 A1
(43) Pub. Date: Aug. 18, 2016

(54) BODYCARE COMPOSITIONS

(71) Applicant: BASF SE, Ludwigshafen (DE)

(72) Inventors: Ivette GARCIA CASTRO, Ludwigshafen (DE); Claudia WOOD, Weinheim (DE); Thomas ZELINSKI, Neuleiningen (DE); Szilard CSIHONY, Gorxheimertal (DE); Wolfgang STAFFEL, Otterstadt (DE); Robert ENGEL, Speyer (DE)

(21) Appl. No.: 15/024,058

(22) PCT Filed: Sep. 29, 2014
(86) PCT No.: PCT/EP2014/070731

§ 371 Date: Mar. 23, 2016

(30) Foreign Application Priority Data
Oct. 9, 2013 (EP) 13187873.8

(51) Int. Cl. A61K 8/81; A61Q 5/02; A61K 8/42; A61Q 19/00; A61K 8/46; A61Q 5/00; A61Q 5/12

(52) U.S. Cl. CPC A61K 8/8147; A61Q 5/00; A61Q 5/02; A61Q 5/12; A61Q 19/00; A61K 8/463; A61K 8/42

(57) ABSTRACT

The present invention relates to care agents comprising ethylene copolymers and to the use of the care agents or of the ethylene copolymers for improving the wet combability of hair and for achieving a waxy, groomed, soft feel to the hair.

The present invention relates to care agents comprising ethylene copolymers and to the use of the care agents or of the ethylene copolymers for improving the wet combability of hair and for achieving a waxy, groomed, soft feel to the hair.

Copolymers comprising ethylene and aminoalkyl acrylates, in copolymerized form, and numerous industrial uses of these copolymers are known.

DE 68903026 (Sumitomo) describes ethylene-d ialkylaminoalkyl acrylate copolymers as sorbents for metallic ions from liquids.

U.S. Pat. No. 3,854,871 (Du Pont) describes the use of ethylene-dialkylaminoalkyl acrylate copolymers for treating textiles.

U.S. Pat. No. 3,928,269 (Phillips) describes the use of ethylene-dialkylaminoalkyl acrylate copolymers for improving the dyeability of polyolefin fibers with acid dyes.

U.S. Pat. No. 3,711,435 (equivalent to DE 2204155) (Du Pont) describes colloidal aqueous dispersions comprising ethylene-aminoalkyl acrylate copolymers, which have been neutralized to at least 40% and use thereof as inter alia coatings for metallic substrates, acid dyeable coatings for cellulose fibers and flocculating agents for purification of water. The copolymers comprise 20 to 80% by weight of ethylene and 80 to 20% by weight of an aminoalkyl acrylate. The neutralization is effected by means of inorganic or organic acids.

EP 331528 (Sumitomo) describes antimicrobial preparations comprising a copolymer as active ingredient, consisting of 40 to 95% by weight of ethylene, 5 to 60% by weight of dialkylaminoalkylacrylamide and up to 20% by weight of further monomers. On page 6, lines 32-36, numerous biocide uses of these copolymers and aqueous dispersions thereof are described. Example E in Table 5 also describes a copolymer comprising also 8% by weight of dimethylaminoethyl methacrylate, in copolymerized form, in addition to ethylene and dimethylaminopropylmethacrylamide.

US 2008/0210561 (Du Pont) describes aqueous cationic polyelectrolyte dispersions comprising a copolymer composed of 70 to 90% by weight of ethylene and 10 to 30% by weight of aminoalkyl (meth)acrylate. For preparing the dispersions, the polymers are dispersed in water with the aid of phosphoric acid and at least one further acid selected from alkylsulfonic acids and gallic acid. The polyelectrolyte dispersions are used as coating compositions for various substrates, for example, for metals, textiles or as hair strengtheners for hair.

WO 2011/018383 describes a process for preparing composite materials wherein modified cellulose fibers are prepared in one step by treating (A) cellulose fibers with aqueous emulsion of (B) at least one ethylene copolymer having a molecular weight Mn of not more than 20 000 g/mol, comprising, as incorporated comonomers, (a) 50 to 95% by weight of ethylene, (b) 5 to 50% by weight of at least one comonomer having at least one alkylated or cycloalkylated amino group, which is linked by a spacer with a polymerizable group, (c) 0 to 30% by weight of further comonomers, and wherein modified cellulose fibers with (C) at least one thermoplastic (D) and optionally one or more additives are admixed.

Modern bodycare compositions, particularly hair shampoos, comprise various types of active ingredients in addition to perfumes and dyes and a series of compounds which increase the shelf-life of the preparations. Besides UV absorbers, vitamins or plant extracts, these also include so-called hair conditioners, which care for the hair and improve its combability and its feel, and increase its shine.

In contrast to most other constituents of shampoos, conditioners attach to the hair and remain there after rinsing. On account of their molecular structure, they position themselves at the damaged sites in the cuticles of the hair and smooth the hair. As a result, the hair becomes less rough and brittle, the hairstyle is given considerably more shine and can be combed more easily. The hair also becomes less sensitive to electrostatic charging. The most important hair conditioning substances in shampoos are the polymeric quaternary ammonium compounds. Cationic cellulose derivatives and polysaccharide derivatives can also be used. Furthermore, silicon compounds are also used for conditioning.

Particularly desired are hair conditioning substances which can be applied with a shampoo and impart to the hair, in addition to the improved combability and the further aforementioned properties, also a waxy, groomed, soft feel.

The term haircare composition refers to compositions according to the invention which care for the hair when used for hair treatment, in particular improve the combability, the cleanliness, the fragrance and the feel to the touch (feel). Hair conditioners are one form of haircare compositions.

In the following, the term “care agent” comprises skin- and haircare compositions, preferably haircare compositions. In the following, these care agents are also referred to as “bodycare compositions”.

The object of the present invention is to provide care agents comprising conditioners which in particular improve the wet combability of the hair.

The present invention relates to a care agent comprising an ethylene copolymer comprising, in copolymerized form,
a) 95 to 50% by weight, preferably 90 to 55% by weight, particularly preferably 80 to 60% by weight of ethylene,
b) 5 to 50% by weight, preferably 10 to 45% by weight, particularly preferably 20 to 40% by weight of at least one monomer of the general formula (I),

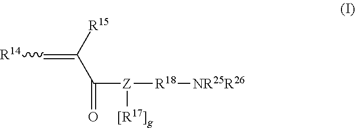

where
R¹⁴ and R¹⁵ are each independently selected from the group consisting of hydrogen, linear or branched C₁-C₈-alkyl, methoxy, ethoxy, 2-hydroxyethoxy, 2-methoxyethoxy and 2-ethoxyethyl,
R¹⁷ is hydrogen or methyl,
R¹⁸ is alkylene or hydroxyalkylene having 1 to 24 carbon atoms, optionally substituted by alkyl, preferably C₂H₄, C₃H₆, C₄H₈, CH₂—CH(OH)—CH₂,
g is 0 or 1,
Z is nitrogen if g=1, or is oxygen if g=0,
R²⁵ and R²⁶ are each independently selected from the group consisting of hydrogen, linear or branched C₁-C₄₀-alkyl, formyl, linear or branched C₁-C₁₀-acyl, N,N-dimethylaminoethyl, 2-hydroxyethyl, 2-methoxyethyl, 2-ethoxyethyl, hydroxypropyl, methoxypropyl, ethoxypropyl and benzyl,
c) 0 to 30% by weight, preferably 0 to 20% by weight of further monomers, where the sum of a), b) and c) is 100% by weight,
   and further conventional ingredients in care agents and thus achieves said object.

The monomer b) can also be present in the cationic form of the general formula (Ia),

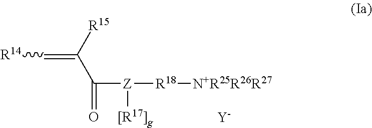

where
R²⁷ is selected from the group consisting of hydrogen, C₁-C₄₀ linear or branched alkyl, formyl, C₁-C₁₀ linear or branched acyl, N,N-dimethylaminoethyl, 2-hydroxyethyl, 2-methoxyethyl, 2-ethoxyethyl, hydroxypropyl, methoxypropyl, ethoxypropyl and benzyl and is preferably selected from methyl and ethyl,
Y⁻ is an anion of an inorganic or organic acid,
the other radicals being analogous to formula (I).

Further subject matters of the present invention and also preferred embodiments are given in the patent claims.

### Monomer b)

Preferred monomers b) are esters of (meth)acrylic acid with amino alcohols, mono- or di-C₁-C₂₄-alkyl substituted on the nitrogen atom. These are particularly preferably selected from the group consisting of N-methylaminoethyl (meth)acrylate, N-ethylaminoethyl (meth)acrylate, N-(n-propyl)aminoethyl (meth)acrylate, N-(n-butyl)aminoethyl (meth)acrylate, N-(tert-butyl)aminoethyl (meth)acrylate, N,N-dimethylaminomethyl (meth)acrylate, N,N-dimethylaminoethyl (meth)acrylate, N,N-diethylaminoethyl (meth)acrylate, N,N-dimethylaminopropyl (meth)acrylate, N,N-diethylaminopropyl (meth)acrylate and N,N-dimethylaminocyclohexyl (meth)acrylate, tert-butylaminoethyl methacrylate, tert-butylaminoethyl acrylate, tert-butylaminopropyl methacrylate, tert-butylaminopropyl acrylate.

In a preferred embodiment of the invention monomer b) is or comprises N,N-dimethylaminoethyl (meth)acrylate, especially preferably N,N-dimethylaminoethyl methacrylate.

One embodiment of the invention refers to haircare compositions comprising ethylene copolymers, which comprise, in copolymerized form, a) 95 to 50% by weight of ethylene, b) 5 to 50% by weight of dimethylaminoethyl methacrylate and c) 0 to 30% by weight of further monomers.

One preferred embodiment of the invention refers to bodycare compositions comprising ethylene copolymers, which comprise, in copolymerized form, a) 80 to 60% by weight of ethylene, b) 20 to 40% by weight of dimethylaminoethyl methacrylate and c) 0 to 20% by weight of further monomers.

Further preferred monomers b) are amides of (meth)acrylic acid with diamines, mono- or di-C₁-C₂₄-alkyl substituted on the nitrogen atom. These are particularly preferably selected from the group consisting of N-[2-(dimethylamino)ethyl]acrylamide, N-[2-(dimethylamino)ethyl]methacrylamide, N-[3-(dimethylamino)propyl]acrylamide, N-[3-(dimethylamino)propyl]methacrylamide, N-[4-(dimethylamino)butyl]acrylamide, N-[4-(dimethylamino)butyl]methacrylamide, N-[2-(diethylamino)ethyl]acrylamide, N-[4-(dimethylamino)cyclohexyl]acrylamide, N-[4-(dimethylamino)cyclohexyl]methacrylamide, N-[8-(dimethylamino)octyl]methacrylamide, N-[12-(dimethylamino)dodecyl]methacrylamide, N-[3-(diethylamino)propyl]methacrylamide and N-[3-(diethylamino)propyl]acrylamide. N-[3-(dimethylamino)propyl]methacrylamide is particularly preferred.

One preferred embodiment of the invention refers to bodycare compositions, in accordance with the invention, comprising ethylene copolymers which are at least partially protonated.

If monomer b) is cationic, the corresponding anion Y⁻ is, for example, sulfate or phosphate, preferably a singly negatively charged anion, for example, halide, particularly chloride or bromide, also hydrogensulfate, C₁-C₄-alkylsulfate, particularly methylsulfate, ethylsulfate, dihydrogenphosphate, formate, acetate, propionate, stearate, palmitate, citrate, tartrate or particularly preferably lactate.

In the case that Y⁻ is selected from anions of polybasic acids, for example sulfate or phosphate, one anion Y⁻ can serve for charge neutralization of more than one equivalent of comonomer (b).

### Monomers c)

All cosmetically acceptable monomers copolymerizable with a) and b), differing from a) and b), are suitable as monomers c).

Examples of possible monomers c) are (meth)acrylates, vinyl esters, vinyl sulfonates, vinyl phosphonates, vinyl ethers, vinyl lactams, e.g. N-vinylpyrrolidone, maleic anhydride, alkylene monomaleates, alkyl dimaleates, in each case having 1 to 18 carbon atoms in the alkyl radical and vinylimidazoles. Particularly suitable monomers c) are also those comprising silicone units (—Si—O—).

The ethylene copolymer contained in the bodycare compositions according to the invention preferably has a number average molecular weight Mₙ of not more than 20 000 g/mol, particularly preferably 1000 to 15 000 g/mol.

It can be prepared by conventional polymerization processes.

The preparation of the ethylene copolymer contained in the bodycare compositions according to the invention is carried out preferably as described in WO 2011/018383, page 8, line 39 to page 9, line 9 for the ethylene copolymer (B).

In an embodiment of the present invention the ethylene copolymer contained in the bodycare compositions according to the invention has a kinematic melt viscosity v in the range of 60 to 150 000 mm²/s, preferably 300 to 90 000 mm²/s, measured at 120° C. according to DIN 51562.

In an embodiment of the present invention the melting point of the ethylene copolymer contained in the bodycare compositions according to the invention is in the range of 40 to 110° C., preferably in the range to 100° C., determined by DSC according to DIN 51007.

In an embodiment of the present invention the density of the ethylene copolymer contained in the bodycare compositions according to the invention is in the range of 0.85 to 0.99 g/cm³, preferably to 0.97 g/cm³, determined according to DIN 53479.

The care agents according to the invention are present preferably in the form of an aqueous emulsion. The preparation of aqueous emulsions of ethylene copolymers contained in the bodycare compositions according to the invention is known per se. The procedure preferably involves placing one or more ethylene copolymers in a vessel, for example, a flask, an autoclave or a tank, and heating the ethylene copolymer(s) and adding one or more Brønsted acids and optionally further substances, for example, water, where the sequence of the addition of Brønsted acid or Brønsted acids and optionally further substances is arbitrary. If it is desirable to prepare the emulsion in question at a temperature above 100° C., it is advantageous to operate at elevated pressure and to select the appropriate vessel. The emulsion produced is homogenized, for example, by mechanical or pneumatic stirring or by shaking. It is advantageous to heat to a temperature above the melting point of the ethylene copolymer. It is advantageous to heat to a temperature of at least 10° C., particularly advantageously to a temperature of at least 30° C., above the melting point of the ethylene copolymer.

Sufficient Brønsted acid can be used so that the ethylene copolymer is present partially, or preferably completely, in neutralized form. In one embodiment of the present invention an excess of Brønsted acid is used.

If monomer b) is polymerized in quaternized form or the ethylene copolymer produced is quaternized, the addition of Brønsted acid can be dispensed with.

In a preferred embodiment of the invention, the ethylene copolymer contained in the bodycare composition is partially or completely neutralized, preferably with acetic acid or lactic acid.

In a preferred embodiment of the invention the amino groups present in the ethylene copolymer are neutralized to 20 to 200%, preferably 40 to 150%, particularly preferably 60 to 120%. The percentages refer to the proportion ratio of acid to amino groups present in the ethylene copolymer. A protonated ethylene copolymer is obtained by the neutralization.

For the preparation of the care agents according to the invention, the ethylene copolymer is preferably provided in the form of an aqueous emulsion. This emulsion preferably has a solids content in the range of 1 to 50, particularly preferably 10 to 30, particularly 15 to 25% by weight.

Haircare compositions according to the invention can be in the form of hair shampoos, hair lotions, hair rinses, hair cures and such like. These compositions and also the bodycare compositions according to the invention can generally further comprise mild surfactants, oil substances, emulsifiers, pearlescent waxes, consistency regulators, thickeners, superfatting agents, stabilizers, polymers, fats, waxes, lecithins, phospholipids, biogenic active ingredients, antioxidants, deodorants, antiperspirants, anti-dandruff compositions, film formers, swelling agents, hydrotropic agents, solubilizers, preservatives, perfume oils, dyes and such like, as further additives and auxiliaries.

Haircare compositions for cleaning of the hair are also referred to as shampoos. It is preferable for the latter to comprise anionic surfactants, particularly preferably at a content of 5 to 20% by weight.

Anionic surfactants which may be used advantageously according to the invention are described in WO 2009/135683, page 4, line 24 to page 5, line 28 and in EP 2 366 377, paragraphs [0014] and [0021].

The haircare compositions according to the invention for cleaning of the hair preferably additionally comprise amphoteric surfactants. Amphoteric surfactants suitable, in accordance with the invention, are described in WO 2009/135683, page 5, lines 30 to 36 and in EP 2366377, paragraph [0022].

Particularly preferred haircare compositions according to the invention for cleaning of the hair include, as amphoteric surfactants, acyl-/dialkylethylenediamines, particularly sodium acylamphoacetate, disodium acylamphodipropionate, disodium alkylamphodiacetate, sodium acylamphohydroxypropylsulfonate, disodium acylamphodiacetate and sodium acyl am phopropionate.

Particularly advantageous shampoos in accordance with the invention comprise a combination of alkyl ether sulfates, particularly lauryl ether sulfate, and cocamidopropyl betaine, particularly preferably alkyl ether sulfates, cocamidopropyl betaine and alkyl polyglycol ester sulfosuccinates, especially preferably lauryl ether sulfate, cocamidopropyl betaine and alkyl polyglycol ester sulfosuccinates.

Shampoos according to the invention particularly advantageously comprise 5-15% by weight, particularly 8-13% by weight of lauryl ether sulfate and 1-5% by weight, particularly 2-4% by weight of cocamidopropyl betaine. Lauryl ether sulfate is a sulfate, based on lauryl alcohol, which has been ethoxylated and/or propoxylated. Cocoamidopropyl betaine is obtainable from the amide of coconut fatty acids and dimethylaminopropylamine by reaction with chloroacetic acid.

Other shampoos according to the invention comprise, in addition to the ethylene copolymer, a combination of 5-15% by weight of lauryl ether sulfate, 1-5% by weight of cocamidopropyl betaine and 1-7% by weight of decyl glucoside.

The shampoos according to the invention can also comprise non-ionic surfactants. Suitable non-ionic surfactants, in accordance with the invention, are described in WO 2009/135683, page 6, lines 4 to 13 and in EP 2 366 377, paragraph [0014].

Haircare compositions in the form of conditioners preferably comprise cationic surfactants, preferably at a content of 0.1 to 2% by weight. Preferred conditioners according to the invention comprise alkylamines, alkylimidazoles, ethoxylated amines, quaternary surfactants or esterquats as cationic surfactants. Further preferred conditioners according to the invention comprise cellulose derivatives, quaternized guar gum derivatives or dimethyldiallylammonium chloride-acrylamide copolymers.

The shampoos according to the invention preferably comprise sodium chloride, PEG-200 hydrogenated glyceryl palmitate, PEG-120 methyl glucose dioleate, PEG-18 glyceryl oleate/cocoate, glyceryl stearate, glyceryl oleate, glyceryl laurate, cocamide MEA, cocamide DEA, laureth-3, laureth-4 or acrylate copolymers (INCI: carbomer, acrylates copolymer, acrylates/beheneth-25 methacrylate copolymer) or hydrophobically modified polyurethanes (INCI: polyurethane-39) as thickeners. Use concentrations of 0.05-5% by weight are particularly advantageous.

In principle, all established, known pearlescent crude materials can be used as pearlescence systems. Suitable pearlescent waxes are described in EP 2366377, paragraph [0014], to which reference is hereby made. The concentrations of pearlescent waxes used to advantage are 0.5-15% by weight.

In place of pearlescence systems an opacifier can also be used. Particularly advantageous is the use of mixtures consisting of glycol distearate, cocoglucoside, glyceryl oleate, glyceryl stearates such as are provided under the trade name Lamesoft®™ Benz (BASF).

A combination of pearlescent agent and opacifier can also advantageously be used.

Haircare compositions according to the invention are also hair rinses and hair cures. In these cases quaternary surfactants are preferably used as surfactants, such as alkylamines, alkylimidazoles, ethoxylated amines or esterquats.

The cationic surfactants used according to the invention can further preferably be selected from the group of the quaternary ammonium compounds, particularly benzyltrialkylammonium chloride, also alkyltrialkylammonium salts, for example, cetyltrimethylammonium chloride or bromide, alkyldimethylhydroxyethylammonium chlorides or bromides, dialkyldimethylammonium chlorides or bromides, alkylamide ethyltrimethylammonium ether sulfates, alkylamidopropyltrimethylammonium chloride, alkylpyridinium salts, for example lauryl- or cetylpyrimidinium chloride, imidazoline derivatives and compounds having cationic character such as amine oxides, for example alkyldimethylamine oxides or alkylaminoethyldimethylamine oxides. Particularly advantageous is the use of cetyltrimethylammonium salts.

Monomeric ammonium compounds are frequently used in hair rinses and hair cures e.g. in concentrations of 0.5-5% by weight. In leave-on products monomeric or polymeric quaternary ammonium compounds are preferably used e.g. in concentrations of 0.1 to 0.5% by weight. These include for example cetrimonium chloride (e.g. Dehyquart®A, BASF) or distearoylethyl hydroxyethylmonium methosulfate (e.g. Dehyquart®F 75, BASF), in addition to other ammonium compounds.

In an embodiment of the invention, the haircare compositions according to the invention comprise styling polymers. Suitable styling polymers according to the invention are mentioned for example in WO 2009/135683, page 9, line 13 to page 10, line 8.

Hair rinses, hair cures and other haircare compositions, also silicones, can be used in shampoos according to the invention for conditioning of hair. Suitable silicone compounds are, for example, dimethylpolysiloxanes, methylphenylpolysiloxanes, cyclic silicones and also amino-, fatty acid-, alcohol-, polyether-, epoxy-, fluorine-, glycoside- and/or alkyl-modified silicone compounds, which may be present at room temperature either as liquids or in resin form. Suitable silicones are, for example, dimethicone and amodimethicone.

In the cosmetic preparations the silicones are typically used in amounts of 0.01 to 5% by weight, preferably 1 to 2% by weight.

In one embodiment of the invention the care agents according to the invention do not comprise silicones.

In an embodiment of the invention the care agents according to the invention also comprise oil substances. Suitable oil substances according to the invention are described in EP 2366377, paragraph [0015], to which reference is hereby made.

In particular, if the care agents according to the invention are present in the form of hair creams and hair cures, said care agents preferably also comprise emulsifiers.

Suitable non-ionogenic emulsifiers according to the invention are described in EP 2 366 377, paragraphs [0016] to [0020].

Suitable anionic emulsifiers according to the invention are described in EP 2366377, paragraph [0021].

Suitable amphoteric and cationic emulsifiers according to the invention are described in EP 2 366 377, paragraph [0022].

In an embodiment of the invention the care agents according to the invention also comprise fats and waxes. Suitable fats and waxes according to the invention are described in EP 2 366 377, paragraph [0023].

Useful consistency regulators for the care agents according to the invention are primarily fatty alcohols or hydroxy fatty alcohols having 12 to 22 and preferably 16 to 18 carbon atoms and, in addition, partial glycerides, fatty acids or hydroxy fatty acids. Suitable consistency regulators according to the invention are described in EP 2 366 377, paragraph [0025].

Metal salts of fatty acids can be used as stabilizers for the care agents according to the invention, such as magnesium, aluminum and/or zinc stearate or ricinoleate.

The care agents according to the invention can also comprise biogenic active ingredients. Such active ingredients are described in EP 2 366 377, paragraph [0030].

The care agents according to the invention can also comprise anti-dandruff compositions. Suitable anti-dandruff compositions are described in EP 2 366 377, paragraph [0032].

To improve the flow characteristics of the care agents, hydrotropic agents may also be used such as ethanol, isopropyl alcohol, or polyols. Suitable hydrotropic agents are described in EP 2 366 377, paragraph [0033].

The present invention also relates to the use of the ethylene copolymers in shampoos, rinses or cures to achieve a better stylability.

In this context, shampoos are haircare compositions comprising cleaning surfactants. These compositions can also be present as 2 in 1 products with additional hair conditioner or also as 2 in 1 products for cleaning of hair and body. In terms of the invention it is unimportant whether it concerns a conventional shampoo, a concentrate or a dry shampoo.

In this context, rinses and cures are compositions for use on the hair, for which the main task is not the cleaning, rather the improvement of combability, feel or other sensory properties of hair and which are typically used on the hair after a shampoo treatment. These compositions may comprise established conditioner substances, such as cationic surfactants, cationic polymers, silicones, silicone copolymers etc. In terms of the invention it is unimportant whether these products are present as solution, gel, balm, lotion, cream, fluid, spray, aerosol, aerosol foam, hydrodispersion, W/O emulsion, O/W emulsion, W/Si emulsion, Si/W emulsion, or other emulsion. It is unimportant whether these compositions are rinsed off or remain on the hair after use.

### EXAMPLES

### Preparation of an Ethylene Copolymer

In a high-pressure autoclave ethylene and N,N-dimethylaminoethyl methacrylate (DMAEMA)

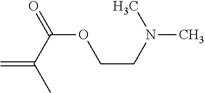

were continuously copolymerized. For this purpose ethylene (12.0 kg/h) was continuously fed into the high-pressure autoclave at the reaction pressure of 1700 bar. Separate from this, the amount of N,N-dimethylaminoethyl methacrylate given in Table 1, optionally diluted with the amount of isododecane given in Table 1, column 5, was initially compressed to an intermediate pressure of 260 bar and fed continuously into the high-pressure autoclave at the reaction pressure of 1700 bar by means of a further compressor. Separate from this, the amount of initiator solution given in Table 1, consisting of tert-amyl peroxypivalate (in isododecane, for concentration see Table 1), was continuously fed into the high-pressure autoclave at the reaction pressure of 1700 bar. Separate from this, the amount of propionaldehyde given in Table 1 was optionally compressed initially to an intermediate pressure of 260 bar and subsequently continuously fed into the high-pressure autoclave at the reaction pressure of 1700 bar by means of a further compressor. The reaction temperature was approximately 220° C. Ethylene copolymers were obtained with the analytical data given in Table 2.

TABLE 1
Preparation of ethylene copolymers contained in the haircare compositions

| | | | | | | PO | | | Copo |
|---|---|---|---|---|---|---|---|---|---|
| | T_{Reactor} | Ethylene | DMAEMA | | PA | in ID | | Conversion | output |
| No. | [° C.] | [kg/h] | [l/h] | ID | [ml/h] | [l/h] | c(PO) | [%] | [kg/h] |
| 1 | 220.6 | 12 | 1.67 | 235 | 235 | 1.64 | 0.05 | 23.0 | 3.2 |
| 2 | 220.2 | 12 | 1.67 | 105 | 105 | 2.21 | 0.05 | 22.7 | 3.1 |
| 3 | 219.8 | 12 | 1.67 | 255 | 15 | 1.70 | 0.06 | 23.0 | 3.2 |
| 4 | 219.8 | 12 | 1.22 | 135 | 135 | 1.14 | 0.06 | 21.2 | 2.8 |
| 5 | 218.6 | 12 | 1.22 | 988 | 52 | 1.35 | 0.06 | 20.0 | 2.7 |
| 6 | 220.8 | 12 | 1.22 | 256 | 13.5 | 1.71 | 0.06 | 21.2 | 2.8 |

T_{Reactor} is understood to mean the maximum internal temperature of the high-pressure autoclave.
Abbreviations:
DMAEMA: N,N-dimethylaminoethyl methacrylate,
PA: propionaldehyde,
ID: isododecane (2,2,4,6,6-pentamethylheptane),
PA in ID: solution of propionaldehyde in isododecane, total volumes of the solution.
PO: tert-amyl peroxypivalate,
c(PO): concentration of PO in ID in mol/l
Copo: ethylene copolymer
The conversion refers to ethylene and is given in % by weight

TABLE 2
Analytical data for the ethylene copolymers

| | Ethylene | DMAEMA | Ethylene | DMAEMA | | | |
|---|---|---|---|---|---|---|---|
| | content | content | content | content | η | Density | |
| No. | [wt %] | [wt %] | [mol %] | [mol %] | [mPa · s] | [g/cm³] | Tₘₑₗₜ [° C.] |
| 1 | 62.8 | 37.2 | 90.4 | 9.6 | 1000 | 0.870 | 48.2 |
| 2 | 62.8 | 37.2 | 90.4 | 9.6 | 2603 | 0.875 | 48.8 |
| 3 | 62.2 | 37.8 | 90.2 | 9.8 | 6357 | 0.882 | 43.2 |
| 4 | 67.8 | 32.2 | 92.1 | 7.9 | 3432 | 0.853 | 40.1 |
| 5 | 67.4 | 32.6 | 92.2 | 7.8 | 7067 | 0.853 | 41.4 |
| 6 | 69.6 | 30.4 | 92.6 | 7.4 | 10645 | 0.868 | 46.7 |

The term “content” is understood to mean the proportion of ethylene in copolymerized form or DMAEMA in the respective ethylene copolymer.
η: dynamic melt viscosity, measured at 120° C. in a plate/cone viscometer (PP 35 Ti) 1.0 mm column, and D = 10 [1/s] according to DIN 53018-1
The content of ethylene and N,N-dimethylaminoethyl methacrylate in the ethylene copolymers was determined by ¹H-NMR spectroscopy.
The density was determined according to DIN 53479. The melting point Tₘₑₗₜ or melting range was determined by DSC (differential scanning calorimetry, differential thermal analysis) according to DIN 51007.

### Preparation of Aqueous Emulsions of Ethylene Copolymers

Emulsions Neutralized with Acetic Acid

### General Preparation Procedure

A 2 liter autoclave with anchor stirrer was charged with the amount of ethylene copolymer given in Table 3 according to example 1. This was heated to 130° C. with stirring and then the amount of 37% by weight of aqueous acetic acid given in Table 3 was added dropwise according to the table over a period of 15 minutes, feed 1. Subsequently the remaining amount of water was added over a period of 30 minutes, feed 2, and stirring continued for a further 15 minutes at 130° C. (external temperature). The external temperature was then lowered to 100° C., the mixture stirred for one hour at 100° C. and then cooled to room temperature over a period of 15 minutes. After filtration using a Perlon filter (100 μm), the respective aqueous emulsions were obtained. Data and properties of the emulsions obtained are summarized in Table 3.

TABLE 3
Acetic acid emulsions of ethylene copolymers

| | | | Amount | | | |
|---|---|---|---|---|---|---|
| | Copo | Glacial acetic | of H₂O | | | |
| | amount | acid [g] in water | [g], | Molar ratio | pH of | Solids content |
| No. | [g] | Feed 1 | Feed 2 | amino groups:acid | emulsion | [wt %] |
| 1 | 225 | 32 g glacial | 800 | 1:1 | 4.9 | 20 |
| | | acetic acid in 69 ml | | | | |
| | | H₂O | | | | |
| 2 | 225 | 32 g glacial | 800 | 1:1 | 4.9 | 20 |
| | | acetic acid in 69 ml | | | | |
| | | H₂O | | | | |
| 3 | 225 | 32 g glacial | 800 | 1:1 | 4.9 | 20 |
| | | acetic acid in 69 ml | | | | |
| | | H₂O | | | | |
| 4 | 225 | 28 g glacial | 800 | 1:1 | 4.6 | 20 |
| | | acetic acid in 72 ml | | | | |
| | | H₂O | | | | |
| 5 | 225 | 27.7 g glacial | 800 | 1:1 | 4.6 | 20 |
| | | acetic acid in | | | | |
| | | 72.2 ml H₂O | | | | |
| 6 | 225 | 26.6 g glacial | 800 | 1:1 | 4.6 | 20 |
| | | acetic acid in | | | | |
| | | 73.4 ml H₂O | | | | |

### Alternative Preparation Procedure

Into a 2 liter autoclave with anchor stirrer were weighed 199.9 g of water with 42.4 g of acetic acid as charge. The mixture was heated to 110° C. (external temperature) with stirring for 30 min. Subsequently, 300 g of an ethylene/DMAEMA copolymer melted at 115° C., prepared according to example 1, were added as rapidly as possible by means of a heatable dropping funnel. At the end of the addition stirring was continued for 10 minutes at approximately 97° C. (internal temperature). Subsequently, 250 g of water were added over 15 min at 130° C. (external temperature) and then 707.7 g of water were added as rapidly as possible. Stirring was then continued at 97° C. (internal temperature) for 2 hours. The emulsion was cooled to 60° C. (internal temperature) and then filtered through a Perlon filter (100 mm).

Emulsions Neutralized with Lactic Acid

### General Preparation Procedure

A 2 liter autoclave with anchor stirrer was charged with the amount of ethylene copolymer given in Table 4, according to example 1. This was heated to 130° C. with stirring and then the amount of 48% by weight or 42% by weight of lactic acid solution given in Table 4 was added dropwise according to the table over a period of 15 minutes, feed 1. Subsequently the remaining amount of water was added over a period of 30 minutes, feed 2, and stirring continued for a further 15 minutes at 130° C. (external temperature). The external temperature was then lowered to 100° C., the mixture stirred for one hour at 100° C. and then cooled to room temperature over a period of 15 minutes. After filtration using a Perlon filter (100 μm), the respective aqueous emulsions were obtained.

TABLE 4
Emulsions of ethylene copolymers neutralized with lactic acid

| | | | Amount | | | |
|---|---|---|---|---|---|---|
| | Copo | Lactic acid [g] in | of H₂O | | | |
| | amount | water | [g], | Molar ratio | pH of | Solids content |
| No. | [g] | Feed 1 | Feed 2 | amino groups:acid | emulsion | [wt %] |
| 1 | 225 | 48 g lactic acid | 800 | 1:1 | 4.9 | 20 |
| | | in 52 ml H₂O | | | | |
| 2 | 225 | 48 g lactic acid | 800 | 1:1 | 4.9 | 20 |
| | | in 52 ml H₂O | | | | |
| 3 | 225 | 48 g lactic acid | 800 | 1:1 | 4.9 | 20 |
| | | in 52 ml H₂O | | | | |
| 4 | 225 | 42 g lactic acid | 800 | 1:1 | 4.6 | 20 |
| | | in 58 ml H₂O | | | | |
| 5 | 225 | 42 g lactic acid | 800 | 1:1 | 4.6 | 20 |
| | | in 58 ml H₂O | | | | |
| 6 | 225 | 42 g lactic acid | 800 | 1:1 | 4.6 | 20 |
| | | in 58 ml H₂O | | | | |

### Alternative Preparation Procedure

Into a 2 liter autoclave with anchor stirrer were weighed 199.9 g of water with 63.6 g of lactic acid as charge. The mixture was heated to 110° C. (external temperature) with stirring for 30 min. Subsequently, 300 g of an ethylene/DMAEMA copolymer melted at 115° C., prepared according to example 1, were added as rapidly as possible by means of a heatable dropping funnel. At the end of the addition stirring was continued for 10 minutes at approximately 97° C. (internal temperature). Subsequently, 250 g of water were added over 15 min at 130° C. (external temperature) and then 707.7 g of water were added as rapidly as possible. Stirring was then continued at 97° C. (internal temperature) for 2 hours. The emulsion was cooled to 60° C. (internal temperature) and then filtered through a Perlon filter (100 mm).

### Shampoo 1

| Initial | | | |
|---|---|---|---|
| weight | | | |
| (g) | Product | Comment (INCI) | SC % |
| 46.5 | Demineralized | | 100 |
| | water | | |
| 0.2 | Jaguar ® C13S | Guar hydroxypropyltrimonium | 100 |
| | | chloride | |
| 5.0 | Polymer 2 | neutralized with 80 mol % lactic acid | 20 |
| 35.7 | Nexapon ® NSO | Sodium laureth sulfate | 29 |
| 12.5 | Tego betaine | Cocoamidopropyl betaine | 30 |
| 0.1 | Euxyl ® K100 | | 100 |
| 1.2 | | Sodium chloride | 100 |

SC: solids content

### Shampoo 2

Formulation analogous to Shampoo 1, except that the polymer 2 was neutralized with 100 mol % lactic acid.

### Shampoo 3

Formulation analogous to Shampoo 1, except that the polymer 2 was neutralized with 120 mol % lactic acid.

All 3 shampoos were optically homogeneous, i.e. there was no sign of phase separation. The haircare compositions according to the invention were also stable in the so-called centrifuge test.

### Viscosity

| | Brookfield viscosity in mPa * s | |
|---|---|---|
| Designation | [Spindle RV-5, 20 rpm] | pH |
| Shampoo 1 | 5440 | 6.84 |
| Shampoo 2 | 8280 | 6.40 |
| Shampoo 3 | 9620 | 5.81 |

Wet Combability of Hair Treated with Haircare Compositions According to the Invention

### Determination of Wet Combability Blank Value

The washed hair was dried overnight in a climate-controlled room at 21° C. and 65% relative humidity. Prior to the measurement, the hair was shampooed twice with Texapon NSO for a total of 1 minute and rinsed for 1 minute, so that it was of defined wetness, i.e. the hair was swollen, and was pressed on filter paper. Prior to the start of the measurement, the tress was pre-combed until knots were no longer present in the hair and thus a constant application of force is necessary for the repeated measurement combing. The tress was then fixed to the support and combed using the finely toothed side of the comb into the finely toothed side of the test comb. The hair was inserted into the test comb for each measurement uniformly and without tension. The measurement was started and evaluated by means of software (TIRA program, TIRA). The individual measurement was repeated 5 to 10 times. The calculated average value was noted, together with the standard deviation.

### Determination of the Measurement Value for Wet Combability:

Following the determination of the blank value, the hair was treated depending on the desired application. The combing force was measured analogously to the blank value determination.

Evaluation: reduction in combing force wet=100−(measurement value*100/blank value); data in %

| | Combability | Combability | |
|---|---|---|---|
| | untreated | treated | |
| | Measurement | Measurement | |
| Shampoo No. | value (BW) | value (BW) | % |
| 1 | 38.3 | 24.8 | 35 |
| 2 | 30.8 | 21.9 | 29 |
| 3 | 37.7 | 23.1 | 39 |

The hair treated with the haircare compositions according to the invention was dried overnight, following the shampoo application, at 21° C. and 65% relative humidity and a manual sensory assessment was undertaken by various trained personnel. Initially, the hair tress was stroked by hand along the direction of the cuticle scales and the smoothness and friction on the surface was noted in comparison to the reference strand. A certain friction phenomenon, which in English is referred to as stick-slip, was established in this case, and refers to the variable transfer of static friction into dynamic friction; in the same way, the strength of this friction phenomenon was assessed by grades, where consumers value more positively a low dynamic friction and low static friction since this makes the hair surface feel smoother, “silkier” and “finer”. Subsequently the hair strand treated with the haircare compositions according to the invention was gripped in the whole hand so that the bundle bends: the softness of the treated hair was established, which is not only a surface property but also a mechanical property of the hair cortex. The cuticle scales were lightly rubbed, for example, with the thumbs, so that a light waxy, looked after feel on the hair treated with the haircare compositions according to the invention was established.

The grading is conducted according to the following principle:

Grade 1=very good, well groomed
Grade 2=good, groomed
Grade 3=satisfactory
Grade 4=not good, weak
Grade 5=unpleasant

Feel assessment of the hair treated with haircare compositions according to the invention

| Shampoo No. | Sensory grade |
|---|---|
| 1 | 1 |
| 2 | 1 |
| 3 | 1 |
| Reference | 3 |
| without wax | |
| emulsion | |

Further examples are the following wash formulations:

| | | Shower | Care | | Care |
|---|---|---|---|---|---|
| Ingredient | INCI | gel | shampoo 4 | Bath foam | shampoo 5 |
| Texapon ® NSO | Sodium laureth | 40 | 25 | 20 | 10 |
| | sulfate | | | | |
| Plantacare ® | Lauryl glycoside | 5 | 10 | 20 | 5 |
| 1200 | | | | | |
| Dehyton ® K | Cocamidopropyl | 10 | | | 8 |
| | betaine | | | | |
| Lanette ® O | Cetearyl alcohol | | 1 | | 1 |
| Lamesoft ® PO65 | Cocoglucoside, | 2.5 | | | |
| | glyceryl oleate | | | | |
| | Guar | | 0.2 | | 0.3 |
| | hydroxypropyl | | | | |
| | trimonium chloride | | | | |
| | Polyquaternium-10 | | 0.2 | | |
| | Polyquaternium-7 | 0.3 | | 0.3 | |
| Dehyquart ® A | Cetrimonium | | 1 | | |
| | chloride | | | | |
| Silicone | Dimethicone or | | 1 | | 1 |
| emulsion | Dimethiconol | | | | |
| Inventive wax | nn | 5 | 5 | 10 | 5 |
| emulsion (20% | | | | | |
| strength) | | | | | |
| Sodium chloride | Sodium chloride | 1 | 1 | 1 | 1.5 |
| Water | Water | to 100 | to 100 | to 100 | to 100 |

The wax emulsions according to the invention, as care agents for skin and hair, are deposited on keratin and cause a hydrophobic effect on the surface which is noticeable as a pleasant waxy, fine feeling and a smoothness.

### Formulation Examples of the Cosmetic Preparation

| | | | WS | | |
|---|---|---|---|---|---|
| | | | Crude | wt % | |
| | | | material | Product | |
| | Substance | INCI | [%] | 8 | 9 |
| 1 | Plantacare ® 818 | Cocoglucoside | 50 | 2 | 2 |
| | UP | | | | |
| 2 | Dehyquart ® L80 | Dicocoylethyl | 100 | 1 | 1 |
| | | hydroxyethyl- | | | |
| | | monium | | | |
| | | methosulfate | | | |
| | | propylene glycol | | | |
| 3 | Lanette ® O | Cetyl stearyl alcohol | 100 | 5 | 5 |
| 4 | Cutina ® GMS | Glyceryl stearate | 100 | 1 | 1 |
| 5 | Copherol ® 1250 C | Tocopheryl acetate | | 0.2 | 0.2 |
| 6 | Dehyquart ® A-CA | Cetrimonium | 25 | 2 | 2 |
| | | chloride | | | |
| 7 | Inventive wax | nn | | 5.0 | 10 |
| | emulsion (20% | | | | |
| | strength) | | | | |
| 8 | Gluadin ® WLM | Hydrolyzed wheat | 20 | 0.5 | 0.5 |
| | | protein | | | |
| 9 | Water dem. | | | to | to |
| | | | | 100 | 100 |

### Examples of Conditioner Formulations

### Example Conditioner Rinse-Off 1

| | | % |
|---|---|---|
| Ingredients | INCI | w/w |
| Phase A | | |
| Deionized Water | Water | to 100 |
| EDETA ® BD | Disodium EDTA | 0.05 |
| | PQ-xy | 0.50 |
| Phase B | | |
| Lanette ® O | Cetyl stearyl alcohol | 3.00 |
| Cremophor ® A6 | Ceteareth-6 (and) stearyl | 2.00 |
| | alcohol | |
| Cremophor ® A20 | Ceteareth-20 | 0.50 |
| Lanette ® 16 | Cetyl alcohol | 1.00 |
| Inventive PE | | 2.00 |
| copolymers | | |
| Luvitol ® Lite | Hydrogenated | 1.50 |
| | polyisobutane | |
| Dow Corning 245 | Cyclopentasiloxane | 3.00 |
| Phase C | | |
| D-Panthenol 75W | Panthenol | 0.50 |
| Propylene Glycol | Propylene glycol | 2.00 |
| Preservative | — | q.s. |
| Perfume | — | q.s. |
| Color | — | q.s. |

The unique effect of ethylene-based copolymeric waxes consists in the fact that keratin-containing surfaces (skin, hair, nails, lashes) or hydroxyapatite-containing surfaces (teeth) can be hydrophobically modified for smoothness. The haircare results in a pleasant smooth feel to the hair. In the case of an aftertreatment, such as styling, relaxing, straightening or hair colouring, the ethylene-based copolymers facilitate performing the process and increase the persistence of the effect. The ethylene copolymers reduce potential damage to the hair which arises during the aftertreatments. These polymers can also be easily washed off if necessary. In general, the ethylene copolymers can be considered as silicone alternatives.

1. A care agent, wherein the care agent is a skincare composition or a haircare composition comprising
A) an ethylene copolymer comprising, in copolymerized form,
a) 95 to 50% by weight of ethylene,
b) 5 to 50% by weight of at least one monomer of the general formula (I) or of the general formula (Ia), 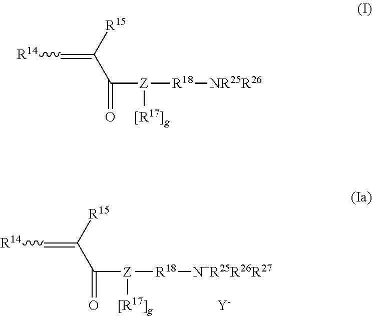
where
R¹⁴ and R¹⁵ are each independently selected from the group consisting of hydrogen, linear or branched C₁-C₈-alkyl, methoxy, ethoxy, 2-hydroxyethoxy, 2-methoxyethoxy and 2-ethoxyethyl,
R¹⁷ is hydrogen or methyl,
R¹⁸ is alkylene or hydroxyalkylene having 1 to 24 carbon atoms, optionally substituted by alkyl,
g is 0 or 1,
Z is nitrogen if g=1, or is oxygen if g=0,
R²⁵ and R²⁶ are each independently selected from the group consisting of hydrogen, linear or branched C₁-C₄₀-alkyl, formyl, linear or branched C₁-C₁₀-acyl, N,N-dimethylaminoethyl, 2-hydroxyethyl, 2-methoxyethyl, 2-ethoxyethyl, hydroxypropyl, methoxypropyl, ethoxypropyl and benzyl,
R²⁷ is selected from the group consisting of hydrogen, C₁-C₄₀ linear or branched alkyl, formyl, C₁-C₁₀ linear or branched acyl, N,N-dimethylaminoethyl, 2-hydroxyethyl, 2-methoxyethyl, 2-ethoxyethyl, hydroxypropyl, methoxypropyl, ethoxypropyl and benzyl,
Y⁻ is an anion of an inorganic or organic acid,
c) 0 to 30% by weight of further monomers,
where the sum of a), b) and c) is 100% by weight, and
B) further conventional ingredients in care agents.

2. The care agent according to claim 1, wherein the ethylene copolymer comprises, in copolymerized form,
a) 80 to 60% by weight of ethylene,
b) 20 to 40% by weight of at least one monomer of the general formula (I) or (Ia)
c) 0 to 20% by weight of further monomers.

3. The care agent according to claim 1,
wherein b) comprises N,N-dimethylaminoethyl methacrylate.

4. The care agent according to claim 1,
wherein the ethylene copolymer is at least partially protonated.

5. The care agent according to claim 1, wherein, if the at least one monomer is of the general formula (Ia), then R²⁷ is hydrogen.

6. The care agent according to claim 1, wherein the care agent is a rinse off care agent.

7. The care agent according to claim 6, wherein the care agent is a haircare composition, comprising
A) the ethylene copolymer in an amount of 0.1 to 5% by weight, and
B) further conventional ingredients in a haircare composition.

8. The care agent according to claim 7, wherein the care agent is a shampoo further comprising
C) anionic surfactants in an amount from 5 to 20% by weight.

9. The care agent according to claim 8, comprising
lauryl ether sulfate in an amount from 5 to 15% by weight.

10. The care agent according to claim 9, comprising
cocamidopropyl betaine in an amount from 1 to 5% by weight.

11. A method for improving the wet combability of hair comprising applying a care agent according to claim 1 to the hair.

12. A method for achieving a waxy, groomed, soft feel to hair, comprising applying a care agent of claim 1 to the hair.

13. The care agent of claim 1 wherein the hair care composition is a shampoo.

14. The care agent of claim 1 wherein R¹⁸ is substituted by an alkyl group selected from the group consisting of C₂H₄, C₃H₆, C₄H₈, and CH₂—CH(OH)—CH₂, and R²⁷ is methyl or ethyl.

15. The care agent according to claim 2, wherein the ethylene copolymer comprises, in copolymerized form,
a) 70 to 60% by weight, of ethylene,
b) 30 to 40 of at least one monomer of the general formula (I) and (Ia), and
c) 0 to 10% by weight, of further monomers.

16. The care agent according to claim 7, comprising
A) the ethylene copolymer in an amount of 0.3 to 3%, by weight.

17. The care agent according to claim 16, comprising
A) the ethylene copolymer in an amount of 0.5 to 2%, by weight.

18. The care agent according to claim 8, comprising
C) anionic surfactant in an amount of 8 to 15%, by weight.

19. The care agent according to claim 9 comprising lauryl ether sulfate in an amount of 8 to 13%, by weight.

20. The care agent according to claim 10 comprising cocamidopropyl betaine in an amount of 2 to 4%, by weight.
